SEQUENCE LISTING (2 sequences)

SEQ ID NO: 1 - DNA, 22 bp, Artificial Sequence
  Description of Artificial Sequencevascular endothelial growth factor (VEGF) cDNA RT-PCR primer
gcctccgaaaccatgaactttc

SEQ ID NO: 2 - DNA, 23 bp, Artificial Sequence
  Description of Artificial Sequencevascular endothelial growth factor (VEGF) cDNA RT-PCR primer
cgagtctgtgtttttgcaggaac

(19) United States
(12) Patent Application Publication
(10) Pub. No.: US 2007/0056049 A1
(43) Pub. Date: Mar. 8, 2007

(54) p53 and VEGF regulate tumor growth of NOS2 expressing cancer cells

(75) Inventors: Stefan Ambs, Newtonville, MA (US); Curtis Harris, Garrett Park, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Govt. of the USA as represented by the Secretary of the Dept. of Health & Human Services, Rockville, MD (US)

(21) Appl. No.: 11/195,006

(22) Filed: Aug. 1, 2005

Related U.S. Application Data
(62) Division of application No. 09/830,977, filed on Jul. 31, 2001, now abandoned.
(60) Provisional application No. 60/109,563, filed on Nov. 23, 1998.

(51) Int. Cl. A61K 49/00; G01N 33/53; A61K 31/198; A61K 31/195

(52) U.S. Cl. 800/3; 435/7.92; 514/565

(57) ABSTRACT

The invention provides, for example, in vitro and in vivo methods for screening modulators of NOS-2 activity using p53 mutant cells, methods of predicting the benefit of administering NOS-2 inhibitors to a cancer patient, and methods of treating cancer by administering NOS-2 inhibitors to patients with p53 mutant cancers.

### CROSS-REFERENCES TO RELATED APPLICATIONS

This application claims priority to U.S. Ser. No. 60/109,563, filed Nov. 23, 1998, herein incorporated by reference in its entirety.

### STATEMENT AS TO FEDERALLY SPONSORED RESEARCH AND DEVELOPMENT

Not applicable.

### FIELD OF THE INVENTION

The present invention provides in vitro and in vivo methods for screening modulators of NOS2 activity using p53 mutant cells, methods of predicting the benefit of administering NOS2 inhibitors to a cancer patient, and methods of treating cancer by administering NOS2 inhibitors to patients with p53 mutant cancers.

### BACKGROUND OF THE INVENTION

Nitric oxide synthase 2 (“NOS2”) is an inducible enzyme that produces nitric oxide (“NO”), a mutagenic and angiogenic molecule (see, e.g., Nguyen et al., Proc. Nat'l. Acad. Sci. U.S.A. 89:3030-3034 (1992); Jenkins et al., Proc. Natl. Acad. Sci. U.S.A. 92:4392-4396 (1995)). High levels of NO induce p53 accumulation in cells, presumably in response to DNA damage, which can lead to p53-mediated growth arrest or apoptosis. In turn, p53, a transcription factor, has been shown to transrepress NOS2 expression in vitro and in vivo (Forrester et al., Proc. Nat'l. Acad. Sci. U.S.A. 93:2442-2447 (1996); Ambs et al., FASEB J. 11:443-448 (1997)); and Ambs et al., Proc. Nat'l Acad. Sci. USA 95:8823-8828 (1998)). p53 therefore is involved repression of NO production via a negative regulatory feedback loop.

Increased expression of inducible nitric oxide synthase (NOS2) has been found in a variety of human cancers, and a NOS2-specific inhibitors can reduce growth of xenografted tumors in mice (Thomsen et al., Br. J. Cancer 72:41-44 (1995); Ellie et al., Neuroreport 7:294-296 (1995); Ambs et al., Cancer Res. 58:334-341 (1998); Gallo et al., J. Natl. Cancer Inst. 90: 587-596 (1998); and Thomsen et al., Cancer Res. 57:3300-3304 (1997)). Recently, NO has been shown to induce vascular endothelial growth factor (“VEGF”) expression in carcinoma cells, leading to tumor neovascularization (Chin et al., Oncogene 15:437-442 (1997)). Thus, the promotion of tumor growth by NO may involve the induction of angiogenic factors (Jenkins et al., Proc. Natl. Acad. Sci. U.S.A. 92:4392-4396 (1995); Edwards et al. J. Surg. Res 63:49-52 (1996); Garcia-Cardena & Folkman, J. Natl. Cancer Inst. 90:560-561 (1998)).

The finding of frequent NOS2 expression in human cancers suggests a pathophysiological role for NO in carcinogenesis. However, the function of NO and NOS2 in carcinogenesis is uncertain. NO has been found to either inhibit or stimulate tumor growth (see, e.g., Dong et al., Cancer Res. 54:789-793 (1994); Jenkins et al., Proc. Natl. Acad. Sci. U.S.A. 92:4392-4396 (1995)). High concentrations of NO are also known to induce cell death in many cell types including tumor cells (Xie et al., J. Exp. Med. 181:1333-1343 (1995); Geng et al., Cancer Res. 56:866-874 (1996); Nicotera et al., Adv. Neuroimmunal. 5:411-420 (1997)), whereas lower concentrations of NO can have an opposite effect and protect against apoptotic cell death from various stimuli (Kim et al., J. Biol. Chem. 272:1402-1411 (1997); Mannick et al., J. Biol. Chem. 272:24125-24128 (1997)). The role of NO and NOS2 in tumor progression, particularly with respect to their interactions with p53, therefore needs to be further defined.

### SUMMARY OF THE INVENTION

To define the role of NO in tumor progression, human carcinoma cell lines were generated that constitutively produced moderate amounts of endogenous NO. The NOS2-expressing cancer cells with wild-type p53 had reduced tumor growth in athymic nude mice, whereas the NOS2-expressing cancer cells with mutated p53 had accelerated tumor growth associated with increased VEGF and neovascularization. The present invention therefore demonstrates that tumor-associated NO production promotes cancer progression by providing a selective growth advantage to cells bearing mutant p53. The effect of moderate NO concentrations on tumor growth is therefore p53 dependent. The present invention thus provides methods of screening for modulators of NOS2 expression in p53 mutant cells, both in vivo and in vitro, as well as methods of predicting the chemotherapeutic benefit of administering NOS2 inhibitors to cancer patients, and methods of treating cancer.

In one aspect, the present invention provides an in vitro method for screening modulators of NOS2 activity, the method comprising the steps of: (i) providing p53 mutant cells that express NOS2; (ii) contacting the cells with compounds suspected of having the ability to modulate NOS2 activity; and (iii) detecting the level of NOS2 expression.

In another aspect, the invention provides an in vivo method for screening modulators of NOS2 activity, the method comprising the steps of: (i) providing p53 mutant cells that express NOS2; (ii) transplanting the cells into a immune deficient animal; (iii) administering to the animal compounds suspected of having the ability to modulate NOS2 activity; and (iv) measuring the growth rate or neovascularization of the tumor.

In one embodiment, the mutant p53 cells express recombinant NOS2.

In one embodiment, the mutant p53 cells produce about 2-15 nmole of nitrate plus nitrite per day.

In one embodiment, the level of NOS2 expression is detected by determining the level of nitrate plus nitrite production using a colorimetric assay.

In one embodiment the level of NOS2 expression is detected by determining the level of VEGF RNA or protein levels.

In one embodiment, the level of NOS2 expression is detected by determining the level of cGMP using RIA or ELISA assays.

In one embodiment, the p53 mutant cells express NOS2 having an activity of from about 3 to about 25 pmole/min/mg.

In one embodiment, the mutant p53 cells are human carcinoma cells. In another embodiment, the mutant p53 cells are selected from the group consisting of HT-29 cells, Calu-6 lung cells, and THLE-5B cells. In another embodiment, the mutant p53 cells have a p53 null mutation, a p53 missense mutation, or inactivated p53 in a complex with SV40 large T antigen.

In one embodiment, the animal is an athymic nude mouse.

In another aspect, the invention provides a method of predicting the benefit of administering NOS2 inhibitors to a cancer patient, the method comprising the step of: determining the p53 status of the patient's tumor or cancer cells; whereby administration of an NOS2 inhibitor to the patient is beneficial when the cancer or tumor cells are p53 mutant cells.

In another aspect, the invention provides a method of treating cancer by administering NOS2 inhibitors to a patient, the method comprising the steps of: (i) determining the p53 status of the patient's cancer or tumor cells and (ii) administering an NOS2 inhibitor to the patient when the cancer or tumor cells are p53 mutant cells.

In one embodiment, the cancer or tumor is selected from the group consisting of breast, brain, head, neck, and colon cancer.

### BRIEF DESCRIPTION OF THE DRAWINGS

FIGS. 1-4: NOS2 expression and tumor growth.

FIGS. 1a and b: NO production in human carcinoma cells does not change cell growth in cell culture. NOS2 expressing Calu-6 and LoVo carcinoma cells were cultured both with and without 2 mM of the NOS2 inhibitor, NG-monomethyl-L-arginine (L-NMA). Clonal cell growth was compared to vector controls (BaglacZ). Each point represents the average clonal growth of 10 colonies per dish in three dishes.

FIG. 2a-d: Tumor probability of NOS2-expressing human carcinoma cells lines is dependent on the p53 status. 3×10⁶ cells of NOS2-expressing Calu-6 and LoVo carcinoma cells, and the vector controls (BaglacZ), were inoculated into 10 athymic nude mice, respectively. NOS2-expressing LoVo cells, which have two wild-type p53 alleles, grow slower (A) and produce smaller tumors (C) than vector controls (BaglacZ). In contrast, NOS2-expressing Calu-6 cells, which lack expression of functional p53, grow faster (B) and produce larger tumors (D) than the vector controls. The NOS2 inhibitor, aminoguanidine (1% AG), suppressed the tumor growth of NOS2-expressing Calu-6 cells (D, *p<0.05, two-tailed Student's t-test) but not vector controls.

FIG. 3: The NOS2 inhibitor aminoguanidine reverses the growth stimulatory effect of NOS2 in tumors of HT-29 colon carcinoma cells. 3×10⁵ cells of NOS2 (Retro-HNOS) and β-galactosidase (BaglacZ) expressing HT-29 cells were inoculated into 40 athymic nude mice, respectively. Half of the animals in both groups received 1% AG in the drinking water. The tumor probability of HT-29 cells is significantly increased by NOS2 when compared to the vector controls (Kaplan-Meier survival analysis: p=0.002). This effect is abolished (p=0.002) through treatment with 1% AG.

FIG. 4a-b: Tumor probability of NOS2 expressing colon carcinoma cells lines correlates with NO production and the p53 status. 5×10⁵ cells of NOS2 expressing HT-29 cell clones and 1×10⁶ cells of NOS2 expressing HCT-116 cell clones were inoculated into 10 athymic nude mice, respectively. The tumor probability of HT-29 cells, which carry a mutant p53, correlates positively with NOS2 activity (A) while the tumor probability of HCT-116 cells, which have wild-type p53, shows an inverse correlation with NOS2 activity (B). The relative NOS2 activity in HT-29 cells (A), measured as nitrite plus nitrate production in cell culture, is 1× for clone 1 (●), 2.7× for clone 2 (▪), and 5× for clone 3 (▴). In HCT-116 cells, the relative activities are 1× in clone 1 (●), 1.5× in clone 2 (▪), and 2× in clone 3 (▴).

FIGS. 5-7: NO induces tumor micro-vascularization and VEGF expression

FIG. 5a-b: Immunohistochemical analysis of the endothelial cell antigen, CD31, in tumors grown by NOS2 (A) or β-galactosidase (B) expressing Calu-6 lung carcinoma cells in athymic nude mice. Numerous capillaries were stained in tumors grown by NOS2-expressing Calu-6 cells (A, arrows). In panel B, scanning magnification shows staining of only one longitudinal section of a large blood vessel (LBV) in tumors grown by the vector control cells; several necrotic areas (NA) are nearby. Number of CD31-positive microvessels per ×250 field: 6.1±2.8 (NOS2) versus 0.7±0.7 (vector control); p<0.01, two-tailed Student's ±-test. Methyl green counterstain. Magnification: A and B, ×100.

FIG. 6: Increased VEGF concentration in protein extracts of NOS2-expressing human carcinoma cells lines. Protein extracts were prepared from RKO, HCT-116, HT-29, Calu-6, and LoVo cells infected with the retroviral construct DFG-iNOS. The NOS2 protein band at 130 kDa was detected by western blot analysis with a polyclonal anti-human NOS2 antibody and 100 μg of protein extract per lane. NOS2 protein was not found in the vector control cell lines (BaglacZ). VEGF protein concentrations were determined after immunoprecipitation of VEGF using 1 mg of protein extract. Molecular size (26-28 kDa) indicates the presence of the membrane-bound VEGF189 splice form. Constitutive expression of VEGF in HCT-116 cells has been reported (Rak et al., Cancer Res. 55:4575-4580 (1995).)

FIG. 7a-b: VEGF protein concentrations are higher (4.3 and 7.1-fold) in the culture medium of NOS2-expressing Calu-6 cells clones than in the culture medium of vector controls (A) and correlate with increased VEGF mRNA expression (B). The NOS-inhibitor L-NMA decreases VEGF secretion. 3×10⁶ cells were cultured in 4 ml of medium for 48 hr±2 mM L-NMA. VEGF was immunoprecipitated out of 1 ml of culture medium. The 4.4 kb VEGF mRNA was detected by northern blotting was a 522 bp ³²P-labeled cDNA (exon 1-7), and the 7.5 kb polycistronic mRNA encoding NOS2 with the full-length human NOS2 cDNA (Geller et al., Proc. Natl. Acad. Sci. U.S.A. 90:3491-3495 (1993); Tzeng et al., Proc. Nat'l Acad. Sci USA 92:11771-11775 (1995)).

### DETAILED DESCRIPTION OF THE INVENTION

### I. INTRODUCTION

The present invention is based on the discovery that tumor-associated NO production promotes cancer progression by providing a selective growth advantage to p53 mutant tumors associated with p53 mutant cells, by providing a selective growth advantage associated with increased VEGF expression and neovascularization. The present invention demonstrates that NOS2-expressing cancer cells with wild-type p53 have reduced tumor growth in athymic nude mice, whereas the NOS2-expressing cancer cells with mutated p53 had accelerated tumor growth associated with increased VEGF and neovascularization. Human breast, brain, head and neck and colon cancers have all been shown to express or overexpress NOS2 (Thomsen et al., Br. J. Cancer 72:41-44 (1995); Ellie et al., Neuroreport 7:294-296 (1995); Ambs et al., Cancer Res. 58:334-341 (1998); Gallo et al., J. Natl. Cancer Inst. 90: 587-596 (1998); and Thomsen et al., Cancer Res. 57:3300-3304 (1997)). Such cancers that express NOS2 and that also have mutated p53 genes would thus have accelerated tumor growth associated with neovascularization.

Furthermore, in the presence of wild-type p53, constitutive expression of NOS2 in those tumors would lead to a p53-mediated growth arrest in the epithelial cells close to the source of NO production. The resulting growth inhibition would provide a strong selection pressure for mutant p53. Indeed, breast, brain, head and neck and colon cancers that overexpress NOS2 have a high frequency of p53 mutations (Greenblatt et al., Cancer Res. 54:4855-4878 (1994)). Clonal selection and growth would be further supported by NO-induced VEGF expression and angiogenesis. Because wild-type p53 transrepresses cytokine-induced NOS2 in a negative feedback loop, NOS2 expression would thus be unchecked in cells with mutant p53 (Forrester et al., Proc. Natl. Acad. Sci. U.S.A. 93:2442-2447 (1996)). However, on the basis of the discoveries provided herein, such p53 mutant cancers could be therapeutically and prophylactically treated with NOS2 inhibitors.

The invention thus provides methods for screening modulators of NOS2 using p53 mutant cells that express NOS2. In one embodiment, modulators of NOS2 activity are screened in vitro, using p53 mutant cells that constitutively or endogenously express NOS2. The cells are contacted with potential NOS2 inhibitors, and the level of VEGF expression is detected, e.g., using PCR, ELISA, or western blot analysis. Alternatively, the level of nitrate plus nitrite production is detected, e.g., using colorimetric methods.

In another embodiment, modulators of NOS2 activity are screened in vivo, using p53 mutant cells that constitutively or endogenously express NOS2. The cells are transplanted into an immune deficient animal such as an athymic nude mouse, and then potential NOS2 modulators are administered to the mouse. Modulation of NOS2 expression is examined, e.g., by measuring tumor growth as compared to untreated control animals.

The invention also provides methods of predicting the chemotherapeutic benefit of administering NOS2 inhibitors to cancer patients, by determining the p53 status and the NOS2 expression pattern of the cancer. If a patient has a cancer that expresses NOS2 and is p53 mutant, then the patient is a candidate for treatment with NOS2 inhibitors. Moreover, the invention also provides methods of treating cancer, by administering NOS2 inhibitors to patients with mutant p53 cancers that express NOS2.

### II. DEFINITIONS

As used herein, the following terms have the meanings ascribed to them unless specified otherwise.

A “p53 mutant cell” refers to a cell that has a p53 negative phenotype, i.e., wild-type p53 is inactivated or is not expressed in the cells. p53 mutant cells can have null or missense gene mutations, where p53 is truncated or elongated, has substituted amino acids, is deleted, or is not expressed, e.g., due to promoter or splice site mutations, etc. p53 mutant cells can also have inactivated p53. For example, in cells that are immortalized with SV40 T antigen, the T antigen binds to and inactivates p53. p53 mutant cells can be transformed cell lines or cells derived from a biopsy or a tissue sample, or cells grown from an explant. The mutant p53 can be a naturally occurring mutation or an induced or engineered mutation.

“NOS2” refers to nitric oxide synthase 2, which is an inducible enzyme that makes nitric oxide. NOS2 protein is typically not detected in cells in the uninduced state (e.g., by ELISA or western blot), and NO production is undetectable. When induced, NOS2 produces nanomolar to micromolar NO concentrations. NOS2 expression is typically induced with cytokines or lipopolysaccharide (see, e.g., Nathan & Xie, Cell 78:915-918 (1994)).

“NOS2 expression” and cells that “express NOS2” refer to a cell that has been induced to express NOS2 (e.g., with cytokines), or a cell that has been engineered to constitutively express NOS2 (e.g., with a nucleic acid encoding recombinant NOS2 operably linked to a constititive promoter). In such a cell, NOS2 is detectable, e.g., by western blot analysis or immunohistochemistry. NOS2 expression therefore refers, e.g., to induction of endogenous NOS2 expression (from its native promoter), induction of exogenous NOS2 (operably linked to its native promoter), constitutive expression of recombinant NOS2 (operably linked to a heterologous promoter), and constitutive expression of endogenous NOS2 (via its native promoter mutated to be constitutive).

Measuring or detecting the level of “NOS2 expression” refers to directly or indirectly detecting the level of NOS2 activity in a cell, e.g., by detecting the level of NOS2 transcription, NOS2 translation, product formation (i.e. NO or nitrate plus nitrite), NOS2 protein activity, activation or inhibition of downstream gene expression (e.g., VEGF, as measured via protein or RNA levels), cGMP level, neovascularization, tumor growth, and the like. In one embodiment, NOS2 expression is determined by measuring NOS2 or downstream gene (i.e., VEGF) RNA levels via PCR or northern blotting. In another embodiment, NOS2 expression is determined by measuring NOS2 or downstream gene (i.e., VEGF) protein production via immunoassay. In another embodiment, NOS2 expression is determined by measuring product (NO) formation using a colorimetric reaction (e.g., using the Griess reagent). In another embodiment, NOS2 expression is measured by detecting cGMP levels via ELISA or RIA. In another embodiment, NOS2 expression is measured via determining neovascularization induced by VEGF expression, with immunohistochemical analysis of the endothelial cell antigen CD31. In another embodiment, NOS2 expression is measured by measuring tumor growth due to VEGF expression and neovascularization.

“Cancer cells” refers to cells that are precancerous, e.g., have genomic mutations that makes the cells susceptible to transformation, or are predisposed to gaining a mutation (e.g., by proximity to NO producing cells) or cells that are cancerous, e.g., transformed and lacking wild-type growth control.

“Tumor cell” refers to precancerous, cancerous, and normal cells in a tumor.

“Cancer patient” refers to a patient with cancer or a patient with a predisposition to cancer, e.g., one that has an inherited mutation that predisposes the patient to developing cancer, e.g., patients with Li-Fraumeni syndrome, ataxia telangiectasia, or adenomatous polyposis.

An “immune deficient animal” refers to an animal, e.g., a mouse, that does not have a normal immune system, e.g., athymic nude mice, SCID mice, or irradiated mice.

“Measuring the growth rate of a tumor” refers to comparing the growth rate of a test tumor (e.g., a tumor treated with an NOS2 modulator) to a control tumor (e.g., a tumor that has not been treated with an NOS2 inhibitor). Tumor growth can be determined by measuring, e.g., diameter, mass, uptake of detectable moieties such as radioactively labeled molecules, tumor markers, and the like.

“Determining p53 status” refers to determining whether a cell has a p53 positive phenotype (e.g., has active, wild-type p53) or a p53 negative phenotype, as described above.

“Determining the level of NOS2 expression” refers to determining whether a cell expresses NOS2, e.g., has the ability to produce nanomolar to micromolar concentrations of NO per day or has detectable NOS2 expression using ELISA or western blotting.

The phrase “modulator of NOS2 activity” in the context of assays for screening compounds that modulate NOS2 includes the determination of any parameter that is indirectly or directly under the influence of NOS2 activity. Such parameters include, e.g., changes in NOS2 RNA or protein levels, changes in NOS2 activity, changes in NO production, changes in nitrate plus nitrite levels, downstream gene expression (i.e., transcriptional and translational induction of VEGF, p53, fos, heme oxygenase-1 or cyclooxygenase-2), neovascularization, tumor growth, reporter gene transcription (luciferase, CAT, β-galactosidase, GFP (see, e.g., Mistili & Spector, Nature Biotechnology 15:961-964 (1997)); signal transduction; phosphorylation and dephosphorylation; receptor-ligand interactions; changes in second messenger concentrations (e.g., cGMP), in vitro, in vivo, and ex vivo. Such functional effects can be measured by any means known to those skilled in the art, e.g., measurement of NOS2 RNA or protein levels, measurement of NOS2 RNA stability, identification of downstream or reporter gene expression (VEGF, p53, CAT, luciferase, β-gal, GFP and the like), e.g., via chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

“Modulators” of NOS2 thus refers to binding, inhibitory or activating molecules for NOS2 activity identified using in vitro and in vivo assays. Inhibitors are compounds that decrease, block, prevent, delay activation, inactivate, desensitize, antagonize, or down regulate NOS2 activity, e.g., aminoguanidine, N^{g}-monomethyl-L-arginine, glucocorticoids, epidermal growth factor, and TGF-β. Activators are compounds that increase, open, activate, facilitate, enhance activation, sensitize, agonize, or up regulate NOS2 activity, e.g., cytokines and lipopolysaccharide. Modulators include genetically modified versions of NOS2, e.g., with altered activity. Modulators are typically peptides, proteins, polypeptides, oligonucleotides, small chemical or organic molecules, and the like. Such assays for modulators and ligands include, e.g., expressing recombinant NOS2 in cells, using cells that have endogenous NOS2 expression, cell extracts with NOS2 expression, tissue explants with NOS2 expression, animals expression NOS2, or providing NOS2 for in vitro reactions, applying putative modulator compounds, and then determining the functional effects on NOS2 activity, as described above.

Samples or assays comprising NOS2 that are treated with a potential modulator are compared to control samples without the modulator to examine the extent of inhibition or activation. Control samples (untreated with the test compound) are assigned a relative NOS2 activity value of 100%. Modulation/inhibition of NOS2 activity is achieved when the NOS2 activity value relative to the control is about 90%, preferably 50%, more preferably 25%. Modulation/activation of NOS2 activity is achieved when the NOS2 activity value relative to the control is 110%, more preferably 150%, more preferable 200% higher.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group., e.g., homoserine, norleucine, methionine sulfoxide, methionine methyl sulfonium Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes (A, T, G, C, U, etc.).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants and alleles of the invention.

The following groups each contain amino acids that are conservative substitutions for one another:

1) Alanine (A), Glycine (G);

2) Serine (S), Threonine (T);

3) Aspartic acid (D), Glutamic acid (E);

4) Asparagine (N), Glutamine (Q);

5) Cysteine (C), Methionine (M);

6) Arginine (R), Lysine (K), Histidine (H);

7) Isoleucine (I), Leucine (L), Valine (V); and

8) Phenylalanine (F), Tyrosine (Y), Tryptophan (W).

(see, e.g., Creighton, Proteins (1984) for a discussion of amino acid properties).

A “label” or a “detectable moiety” is a composition detectable by spectroscopic, photochemical, biochemical, immunochemical, or chemical means. For example, useful labels include 32P, fluorescent dyes, electron-dense reagents, enzymes (e.g., as commonly used in an ELISA), biotin, digoxigenin, or haptens and proteins for which antisera or monoclonal antibodies are available (e.g., a polypeptide can be made detectable, e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein). See, e.g., Ausubel, supra, for an introduction to recombinant techniques.

A “promoter” is defined as an array of nucleic acid control sequences that direct transcription of a nucleic acid. As used herein, a promoter typically includes necessary nucleic acid sequences near the start site of transcription, such as, in the case of a polymerase II type promoter, a TATA element. As used herein, a promoter also optionally includes distal enhancer or repressor elements, which can be located as much as several thousand base pairs from the start site of transcription. The promoters often have an element that is responsive to transactivation by a DNA-binding moiety such as a polypeptide, e.g., Gal4, the lac repressor and the like. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes an “expression cassette,” which comprises a nucleic acid to be transcribed operably linked to a promoter.

“Antibody” refers to a polypeptide substantially encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′2, a dimer of Fab which itself is a light chain joined to V_{H}—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially an Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies or those synthesized de novo using recombinant DNA methodologies (e.g., single chain Fv).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, and/or quantify the antigen.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

### III. ASSAYS FOR MODULATORS OF NOS2

A. Assays for NOS2 Activity

Assays for NOS2 activity can be used to test for ligands, inhibitors, and activators of NOS2, which can then be used to modulate NOS2 expression and downstream VEGF expression associated with neovascularization. Such modulators are useful as prophylactic and therapeutic agents for cancer. Furthermore, modulation of NOS2 expression can be used to lower local NO levels and reduce the incidence of p53 mutations in adjacent epithelial cells. The activity of NOS2 can be assessed using a variety of in vitro and in vivo assays, by measuring, e.g., NOS2 protein activity, NOS2 protein or mRNA levels, NO production, VEGF activity, VEGF protein or mRNA levels, tumor growth; transcriptional activation or repression of a reporter gene; second messengers levels (e.g., cGMP); cytokine, and hormone production levels; using e.g., immunoassays, hybridization assays, colorimetric assays, amplification assays, enzyme activity assays, and the like.

Modulators of NOS2 activity are tested in p53 mutant cells using biologically active NOS2 and fragments thereof, either recombinant or naturally occurring. NOS2 can be recombinantly expressed in a cell, naturally expressed in a cell, recombinantly or naturally expressed in cells transplanted into an animal, or recombinantly or naturally expressed in a transgenic animal. Modulation is tested using one of the in vitro or in vivo assays described herein. Samples or assays that are treated with a potential NOS2 inhibitor or activator are compared to control samples without the test compound, to examine the extent of modulation or ligand binding. Control samples (untreated with activators or inhibitors) are assigned a relative NOS2 activity value of 100. Inhibition of NOS2 is achieved when the NOS2 activity value relative to the control is about 90%, preferably 50%, more preferably 25%. Activation of NOS2 is achieved when the NOS2 activity value relative to the control is 110%, more preferably 150%, more preferably 200% higher.

Generally, the compounds to be tested are present in the range from 0.1 nM to 10 mM. A known inhibitor of NOS2 activity, e.g., aminoguanidine, can be used as a positive control. Cells that have p53 null mutations, p53 missense mutations, or inactivation of p53 (e.g., with SV40 T antigen) are used in the assays of the invention, both in vitro and in vivo. Suitable cultured cells that are p53 mutant include HT-29 cells, CaLu-6 lung cells, and THLE-5B cells. Preferably, human cells are used. Cell lines can also be created or isolated from tumors that have mutant p53. Optionally, the cells can be transfected with an exogenous NOS2 gene operably linked to a constitutive promoter, to provide higher levels of NOS2 expression. Alternatively, endogenous NOS2 levels can be examined. The cells can be treated to induce NOS2 expression. The cells can be immobilized, be in solution, be injected into an animal, or be naturally occurring in a transgenic or non-transgenic animal.

The effects of the test compounds upon the function of the NOS2 polypeptides can be measured by examining any of the parameters described above. Any suitable physiological change that affects NOS2 activity can be used to assess the influence of a test compound on the polypeptides of this invention. When the functional consequences are determined using intact cells or animals, one can also measure a variety of effects such as tumor growth, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian NOS2 is used, e.g., mouse NOS2, preferably human NOS2.

Preferred assays for NOS2 activity can be performed in vitro. In each assay, NOS2 is first contacted with a potential modulator and incubated for a suitable amount of time, e.g., from 0.5 to 48 hours. In one preferred in vitro assay format, NOS2 expression in cultured cells is measured by examining NO production (see Example I). The level of NO production is determined using a colorimetric reaction with the Griess reagent. In this assay, nitrate in the culture medium is first converted to nitrite using E. coli nitrate reductase, and then the level of nitrite is determined with the Griess reagent (Forrester et al., Proc. Nat'l Acad. Sci. U.S.A. 93:2442-2447 (1996)). The test sample is compared to control cells untreated with the modulator.

In another embodiment, NOS2 levels are determined in vitro by measuring the level of NOS2 protein or mRNA. The level of NOS2 protein is measured using immunoassays such as western blotting, ELISA and the like with an NOS2 specific antibody. For measurement of mRNA, amplification, e.g., using PCR, LCR, or hybridization assays, e.g., northern hybridization, RNAse protection, dot blotting, are preferred. VEGF protein and mRNA levels can be measured in the same fashion (see, e.g., Example IV). The level of protein or mRNA is detected using directly or indirectly labeled detection agents, e.g., fluorescently or radioactively labeled nucleic acids, radioactively or enzymatically labeled antibodies, and the like, as described herein.

In yet another assay, cGMP levels in a cell can be used in vitro to test for NOS2 modulation. NOS2 is known to induce cGMP levels (see, e.g., Felley-Bosco et al., Am J. Respir. Cell. Biol. 11:159-164 (1994)). After treatment of the cell with NOS2 modulators, inhibition or activation of NOS2 can be measured by determining the level of cGMP as compared to a control. Commercially available ELISA assays for cGMP can be used in such an assay.

Alternatively, a reporter gene system can be devised using the NOS2 promoter operably linked to a reporter gene such as luciferase, green fluorescent protein, CAT, or β-gal. The reporter construct is typically transfected into a p53 mutant cell. After treatment with a potential NOS2 modulator, the amount of reporter gene transcription, translation, or activity is measured according to standard techniques known to those of skill in the art.

Another example of a preferred assay format useful for monitoring NOS2 activity is performed in vivo. In this assay (described in Example II), cultured p53 mutant cells that express or overexpress NOS2 (as described above) are injected subcutaneously into an immune compromised mouse such as an athymic mouse, an irradiated mouse, or a SCID mouse. NOS2 modulators are administered to the mouse, e.g., a chemical ligand library. After a suitable length of time, preferably 4-8 weeks, tumor growth is measured, e.g., by volume or by its two largest dimensions, and compared to the control. Tumors that have statistically significant reduction (using, e.g., Student's T test) are said to have inhibited growth. Alternatively, the extent of tumor neovascularization can also be measured, as described in Example III. Immunoassays using endothelial cell specific antibodies are used to stain for vascularization of the tumor and the number of vessels in the tumor. Tumors that have a statistically significant reduction in the number of vessels (using, e.g., Student's T test) are said to have inhibited neovascularization.

B. Computer Assisted Drug Design

Yet another assay for compounds that modulate NOS2 activity involves computer assisted drug design, in which a computer system is used to generate a three-dimensional structure of NOS2 based on the structural information encoded by the amino acid sequence. Using this system, potential modulators are identified and then tested using the in vitro and in vivo assays described above. The input amino acid sequence interacts directly and actively with a pre-established algorithm in a computer program to yield secondary, tertiary, and quaternary structural models of the protein. The models of the protein structure are then examined to identify regions of the structure that have the ability to bind, e.g., ligands. These regions are then used to identify potential ligands and modulators of NOS2 activity. The nucleotide and amino acid sequence of NOS2 is known and can be obtained from publicly available databases (see, e.g., Geller et al., Proc. Nat'l Acad. Sci USA 90:3491-3495 (1993)).

The three-dimensional structural model of the protein is generated by entering protein amino acid sequences of at least 10 amino acid residues or corresponding nucleic acid sequences encoding a NOS2 polypeptide into the computer system. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the NOS2 protein to identify ligands that bind to NOS2. Binding affinity between the protein and ligands is determined using energy terms to determine which ligands have an enhanced probability of binding to the protein.

C. Modulators

The compounds tested as modulators of NOS2 can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

As noted, the invention provides solid phase based in vitro assays in a high throughput format, where the mutant p53 cell expressing NOS2 is attached to a solid phase substrate. Control reactions that measure the expression level of the selected RNA in a reaction that does not include a transcription modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions which do not include a modulator provide a background level of expression from a given coding DNA.

In some assays it will be desirable to have controls to ensure that the components of the assays are working properly. For example, a known inhibitor of NOS2 such as aminoguanidine can be added, and the resulting inhibition of NOS2 detected.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay several different plates per day; assay screens for up to about 6,000-20,000 different compounds is possible using the integrated systems of the invention. More recently, microfluidic approaches to reagent manipulation have been developed, e.g., by Caliper Technologies (Palo Alto, Calif.).

### IV. ISOLATION OF NUCLEIC ACIDS

A. General Recombinant DNA Methods

Polypeptides and nucleic acids, e.g., NOS2, are used in the assays described above. For example, recombinant NOS2 can be used to produce cells that constitutively express NOS2. Such polypeptides and nucleic acids can be made using routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-products, inc. (http://www.htibio.com), BMA Biomedicals Ltd (U.K.), Bio.Synthesis, Inc., and many others.

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides can be chemically synthesized according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983). The sequence of the cloned genes and synthetic oligonucleotides can be verified after cloning using, e.g., the chain termination method for sequencing double-stranded templates of Wallace et al., Gene 16:21-26 (1981). Again, as noted above, companies such as Operon Technologies, Inc. provide an inexpensive commercial source for essentially any oligonucleotide.

B. Cloning Methods

In general, the nucleic acid sequences encoding genes of interest, such as NOS2, p53 or VEGF and related nucleic acid sequence homologs, are cloned from cDNA and genomic DNA libraries by hybridization with a probe, or isolated using amplification techniques with oligonucleotide primers. Preferably mammalian, more preferably human sequences are used. For example, NOS2 sequences are typically isolated from mammalian nucleic acid (genomic or cDNA) libraries by hybridizing with a nucleic acid probe, the sequence of which can be derived from Geller et al., supra. A suitable tissue from which human NOS2 RNA and cDNA can be isolated is hepatocytes or cultured DLD-1 human colon carcinoma cells treated with cytokines.

Amplification techniques using primers can also be used to amplify and isolate, e.g., a nucleic acid encoding NOS2, from DNA or RNA (see, e.g., Dieffenfach & Dveksler, PCR Primer: A Laboratory Manual (1995)). These primers can be used, e.g., to amplify either the full length sequence or a probe of one to several hundred nucleotides, which is then used to screen a mammalian library for the full-length nucleic acid of choice. Nucleic acids can also be isolated from expression libraries using antibodies as probes. Such polyclonal or monoclonal antibodies can be raised, e.g., using the sequence of NOS2.

Polymorphic variants and alleles that are substantially identical to the gene of choice can be isolated using nucleic acid probes, and oligonucleotides under stringent hybridization conditions, by screening libraries. Alternatively, expression libraries can be used to clone, e.g., NOS2 and NOS2 polymorphic variants and alleles, by detecting expressed homologs immunologically with antisera or purified antibodies made against NOS2, which also recognize and selectively bind to the NOS2 homolog.

To make a cDNA library, one should choose a source that is rich in the mRNA of choice, e.g., for human NOS2 mRNA, hepatocytes or DLD-1 human colon carcinoma cells treated with cytokines. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in non-lambda expression vectors. These vectors are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating a nucleic acid and its homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of, e.g., NOS2 directly from mRNA, from cDNA, from genomic libraries or cDNA libraries. Degenerate oligonucleotides can be designed to amplify NOS2 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of NOS2 encoding mRNA in physiological samples, for nucleic acid sequencing, or for other purposes. Genes amplified by the PCR reaction can be purified from agarose gels and cloned into an appropriate vector.

As described above, gene expression of NOS2, p53 or VEGF can also be analyzed by techniques known in the art, e.g., reverse transcription and PCR amplification of mRNA, isolation of total RNA or poly A+ RNA, northern blotting, dot blotting, in situ hybridization, RNase protection, probing DNA microchip arrays, and the like. All of these techniques are standard in the art.

Synthetic oligonucleotides can be used to construct recombinant genes for use as probes or for expression of protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the NOS2 nucleic acid. The specific subsequence is then ligated into an expression vector.

The nucleic acid encoding the protein of choice is typically cloned into intermediate vectors before transformation into prokaryotic or eukaryotic cells for replication and/or expression. These intermediate vectors are typically prokaryote vectors, e.g., plasmids, or shuttle vectors. Optionally, cells can be transfected with recombinant NOS2 operably linked to a constitutive promoter, to provide higher levels of NOS2 expression in cultured cells (see, e.g., Example I).

C. Expression in Prokaryotes and Eukaryotes

To obtain high level expression of a cloned gene or nucleic acid, such as those cDNAs encoding NOS2, one typically subclones NOS2 into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the NOS2 protein are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known in the art and are also commercially available.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. The promoter typically cam also include elements that are responsive to transactivation, e.g., hypoxia responsive elements, Gal4 responsive elements, lac repressor responsive elements, and the like. The promoter can be constitutive or inducible, heterologous or homologous.

In addition to the promoter, the expression vector typically contains a transcription unit or expression cassette that contains all the additional elements required for the expression of the nucleic acid in host cells. A typical expression cassette thus contains a promoter operably linked, e.g., to the nucleic acid sequence encoding NOS2, and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical (one expression vector is described in Example I). Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a NOS2 encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

After the expression vector is introduced into the cells, the transfected cells are cultured under conditions favoring expression of the protein.

### V. PROTEIN PURIFICATION

If necessary, naturally occurring or recombinant proteins can be purified for use in functional assays, e.g., to make antibodies to detect NOS2, p53 or VEGF. Naturally occurring NOS2 is purified, e.g., from mammalian tissue such as liver tissue. Recombinant NOS2, p53, or VEGF are purified from any suitable expression system, e.g., by expressing NOS2 in E. coli and then purifying the recombinant protein via affinity purification, e.g., by using antibodies that recognize a specific epitope on the protein or on part of the fusion protein, or by using glutathione affinity gel, which binds to GST. In some embodiments, the recombinant protein is a fusion protein, e.g., with GST or Gal4 at the N-terminus.

The protein of choice may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant protein is being purified. For example, proteins having established molecular adhesion properties can be reversible fused to NOS2. With the appropriate ligand, NOS2 can be selectively adsorbed to a purification column and then freed from the column in a relatively pure form. The fused protein is then removed by enzymatic activity. Finally, NOS2 could be purified using immunoaffinity columns.

A. Purification of Protein from Recombinant Bacteria

Recombinant proteins are expressed by transformed bacteria in large amounts, typically after promoter induction; but expression can be constitutive. Promoter induction with IPTG is a one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The protein of choice is separated from other bacterial proteins by standard separation techniques, e.g., with Ni—NTA agarose resin.

Alternatively, it is possible to purify the recombinant protein from bacteria periplasm. After lysis of the bacteria, when the protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the protein, e.g., NOS2 can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The protein of choice can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### VI. IMMUNOLOGICAL DETECTION OF PROTEINS

In addition to the detection of NOS2, p53, and VEGF genes and gene expression using nucleic acid hybridization technology, one can also use immunoassays to detect NOS2, p53, and VEGF or to measure NOS2 or VEGF activity, e.g., to identify modulators of NOS2 activity. Immunoassays can be used to qualitatively or quantitatively analyze NOS2, p53, and VEGF. A general overview of the applicable technology can be found in Harlow & Lane, Antibodies: A Laboratory Manual (1988).

A. Antibodies

Methods of producing polyclonal and monoclonal antibodies that react specifically with VEGF, p53, and NOS2 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). In addition, as noted above, many companies, such as BMA Biomedicals, Ltd., HTI Bio-products, and the like, provide the commercial service of making an antibody to essentially any peptide.

A number of VEGF, p53, and NOS2 comprising immunogens may be used to produce antibodies specifically reactive with VEGF, p53, or NOS2. For example, recombinant NOS2 or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. To improve reproducibility, an inbred strain of mice (e.g., BALB/C mice) can be immunized to make the antibody; however, standard animals (mice, rabbits, etc.) used to make antibodies are immunized with the protein using a standard adjuvant, such as Freund's adjuvant, and a standard immunization protocol (see Harlow & Lane, supra). The animal's immune response to the immunogen preparation is monitored by taking test bleeds and determining the titer of reactivity to the protein of choice. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-VEGF or NOS2 proteins or even other related proteins, e.g., from other organisms, using a competitive binding immunoassay. Specific polyclonal antisera and monoclonal antibodies will usually bind with a KD of at least about 0.1 mM, more usually at least about 1 μM, preferably at least about 0.1 μM or better, and most preferably, 0.01 μM or better.

Once VEGF, p53, or NOS2 specific antibodies are available, these proteins can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

VEGF, p53, or NOS2 can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case the VEGF, NOS2 or antigenic subsequence thereof). The antibody may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled NOS2, p53, or VEGF polypeptide or a labeled anti-NOS2, p53, or VEGF antibody. Alternatively, the labeling agent may be a third moiety, such a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol. 111:1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-Competitive Assay Formats

Immunoassays for detecting VEGF, p53, or NOS2 in samples may be either competitive or noncompetitive. Noncompetitive immunoassays are assays in which the amount of antigen is directly measured. In one preferred “sandwich” assay, for example, the anti-antigen antibodies can be bound directly to a solid substrate on which they are immobilized. These immobilized antibodies then capture antigen present in the test sample. Antigen thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive Assay Formats

In competitive assays, the amount of NOS2, p53, or VEGF present in the sample is measured indirectly by measuring the amount of a known, added (exogenous) antigen displaced (competed away) from an anti-antigen antibody by the unknown antigen present in a sample. In one competitive assay, a known amount of antigen is added to a sample and the sample is then contacted with an antibody that specifically binds to the antigen. The amount of exogenous antigen bound to the antibody is inversely proportional to the concentration of antigen present in the sample. In a particularly preferred embodiment, the antibody is immobilized on a solid substrate. The amount of antigen bound to the antibody may be determined either by measuring the amount of antigen present in an antigen/antibody complex, or alternatively by measuring the amount of remaining uncomplexed protein. The amount of antigen may be detected by providing a labeled antigen molecule.

A hapten inhibition assay is another preferred competitive assay. In this assay the known antigen is immobilized on a solid substrate. A known amount of anti-antigen antibody is added to the sample, and the sample is then contacted with the immobilized antigen. The amount of anti-antigen antibody bound to the known immobilized antigen is inversely proportional to the amount of antigen present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Cross-Reactivity Determinations

Immunoassays in the competitive binding format can also be used for crossreactivity determinations. For example, an NOS2, p53, or VEGF protein can be immobilized to a solid support. Proteins are added to the assay that compete for binding of the antisera to the immobilized antigen. The ability of the added protein to compete for binding of the antisera to the immobilized protein is compared to the ability of antigen to compete with itself. The percent crossreactivity for the above proteins is calculated, using standard calculations. Those antisera with less than 10% crossreactivity with the added protein are selected and pooled. The cross-reacting antibodies are optionally removed from the pooled antisera by immunoabsorption with the added protein.

The immunoabsorbed and pooled antisera are then used in a competitive binding immunoassay as described above to compare a second protein, thought to be perhaps an allele or polymorphic variant of NOS2, p53, or VEGF, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the first protein that is required to inhibit 50% of binding, then the second protein is said to specifically bind to the polyclonal antibodies generated to the immunogen of choice.

Other Assay Formats

Western blot (immunoblot) analysis is used to detect and quantify the presence of NOS2, p53, or VEGF in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind NOS2. The anti-antigen antibodies specifically bind to the antigen on the solid support. These antibodies may be directly labeled or alternatively may be subsequently detected using labeled antibodies (e.g., labeled sheep anti-mouse antibodies) that specifically bind to the anti-antigen antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a specific protein, or secondary antibodies that recognize antibodies to the specific protein.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### VII. TRANSGENIC MICE

Transgenic mice constitutively expressing NOS2, or with mutant p53, can be made by simple insertion of NOS2 or a mutated version of p53 into the mouse genome or by homologous recombination, in a pluripotent cell line that is capable of differentiating into germ cell tissue. For example, DNA construct that contains constitutively expressed NOS2 is introduced into the nuclei of embryonic stem cells. In a portion of the cells, the introduced DNA recombines with the endogenous copy of the mouse gene, replacing it with the human copy. Alternatively, cells can be selected that express both the endogenous and human genes. In addition, knock-out mice can be made with a p53 negative phenotype, where the endogenous p53 gene is replaced by a marker gene such as neo. Missense p53 mice also can be made using homologous recombination.

Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)).

Cells and animals that have one or more functionally disrupted endogenous genes or that express an exogenous gene have various commercial applications. For example, a transgenic mouse that is heterozygous or homozygous for integrated transgenes that have functionally disrupted the endogenous p53 gene can be used as a sensitive in vivo screening assay for modulators of NOS2 activity. Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

### VIII. KITS

Kits for detection of NOS2, p53, and VEGF are provided by the present invention. Such kits contain NOS2, p53, and VEGF specific reagents that specifically hybridize to NOS2, p53, or VEGF nucleic acid, such as specific probes and primers, and NOS2, p53, or VEGF specific reagents that specifically bind to the protein of choice, e.g., antibodies. The kits are in the assays described herein for identification of modulators of NOS2, or for screening patients to determine their p53 and NOS2 status prior to treatment with NOS2 inhibitors.

Nucleic acid assays for the presence of NOS2, p53, or VEGF DNA and RNA in a sample include numerous techniques are known to those skilled in the art, such as Southern analysis, northern analysis, dot blots, RNase protection, S1 analysis, amplification techniques such as PCR and LCR, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, NOS2 or VEGF protein can be detected with the various immunoassay techniques described above. The test sample is typically compared to both a positive control (e.g., a sample expressing recombinant NOS2) and a negative control.

The present invention also provides for kits for screening for modulators of NOS2. Such kits can be prepared from readily available materials and reagents. For example, such kits can comprise any one or more of the following materials: Vectors comprising NOS2, reaction tubes, antibodies for VEGF or NOS2, oligos for VEGF or NOS2, and instructions for testing NOS2 activity after application of a potential modulator compound. A wide variety of kits and components can be prepared according to the present invention, depending upon the intended user of the kit and the particular needs of the user. For example, the kit can be tailored for in vitro or in vivo assays for measuring the activity of NOS2.

### IX. USE OF NOS2 AND p53 LEVELS TO PREDICT BENEFIT OF CHEMOTHERAPY

As described above, NOS2 inhibitors are useful both as therapeutic agents and as prophylactic agents for patients that have cancers with mutated p53, or for patients who are predisposed to developing cancers with mutant p53. Such cancers are susceptible to growth reduction, by preventing VEGF production and neovascularization, using NOS2 inhibitors.

In order to determine whether a patient would benefit from administration of an NOS2 inhibitor, the p53 status of the tumor, cancer, or precancerous cells must first be established. For prophylactic uses, the likelihood of developing a p53 negative phenotype should be established, e.g., whether the patient has cells that are heterozygous for wild-type p53 or has a genetic condition linked to loss of p53 in certain tissues, e.g., Li Fraumeni syndrome and the like. For therapeutic and prophylactic uses, a sample of the patient's precancer, tumor or cancer cells is obtained by means known to those skilled in the art, e.g., biopsy etc. The p53 status of the patient's cells can be established by any of the means described above. Typically a sample of a suitable cell type, e.g., head, neck, breast, brain, or colon, is obtained. The p53 phenotype is determined using p53 specific reagents that detect p53 DNA, RNA or protein, as described above.

The level of NOS2 expression in the patient's cells can optionally be determined. However, even low levels of NOS2 expression can promote mutagenesis, tumor growth, and neovascularization. Thus, patients with p53 negative cancer or tumor cells would likely benefit from administration of NOS2 inhibitors, whether or not NOS2 expression is detectable in the cells.

As described above, NOS2 expression can be determined by examining, e.g., NOS2 protein levels, RNA levels, or NO production. The sample is compared to an adjacent tissue control, or to a control cell that does not express NOS2 (e.g., NOS2 expression is not induced or is not constititive). Typically even a low to moderate level of NOS2 expression provides an indication that the patient would benefit from treatment with NOS2 inhibitors. Low to moderate levels of NOS2 expression are typically determined by examining, e.g., NO production (nanomolar to micromolar concentrations per day), or by detecting the presence of NOS2 protein via immunoassay techniques such as ELISA and western blot analysis. As described above, even levels of NOS2 that are not detectable above background can provide neovascularization, tumor growth, and mutagenesis properties to a cancer or tumor.

Once the p53 status and optionally the level of NOS2 expression have been established, a decision is made whether to administer the NOS2 inhibitors. As described above, a negative 53 phenotype would indicate that a patient would benefit from administration of NOS2 inhibitors for either prophylactic or therapeutic treatments, either to prevent development of tumor or cancer cells, or to slow down, stop, or reduce the growth of pre-existing tumor or cancer cells.

### X. THERAPEUTIC USES OF NOS2 INHIBITORS

NOS2 modulators can be administered directly to the patient for inhibition of cancer, tumor, or precancer cells in vivo. Administration is by any of the routes normally used for introducing a modulator compound into ultimate contact with the tissue to be treated. The NOS2 modulators are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The NOS2 modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient, in the context of the present invention should be sufficient to effect a beneficial therapeutic response in the patient over time. The dose will be determined by the efficacy of the particular NOS2 modulators employed and the condition of the patient, as well as the body weight or surface area of the patient to be treated. The size of the dose also will be determined by the existence, nature, and extent of any adverse side-effects that accompany the administration of a particular compound or vector in a particular patient

In determining the effective amount of the modulator to be administered in the treatment or prophylaxis of cancer, the physician evaluates circulating plasma levels of the modulator, modulator toxicities, progression of the disease, and the production of anti-modulator antibodies. In general, the dose equivalent of a modulator is from about 1 ng/kg to 10 mg/kg for a typical patient. Administration of NOS2 inhibitors is well known to those of skill in the art (see, e.g., Bansinath et al., Neurochem Res. 18:1063-1066 (1993); Iwasaki et al., Jpn. J. Cancer Res. 88:861-866 (1997); Tabrizi-Rad et al., Br. J. Pharmacol. 111:394-396 (1994)).

For administration, inhibitors of the present invention can be administered at a rate determined by the LD-50 of the inhibitor, and the side-effects of the inhibitor at various concentrations, as applied to the mass and overall health of the patient. Administration can be accomplished via single or divided doses.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### IX. EXAMPLES

The following examples are provided by way of illustration only and not by way of limitation. Those of skill in the art will readily recognize a variety of noncritical parameters that could be changed or modified to yield essentially similar results.

### Example I

### Constitutive Expression of NOS2 in Human Carcinoma Cell Lines

A. Methods

Retroviral gene transfer of human NOS2: Human carcinoma cells were infected, as described, with either the retroviral vector DFG-iNOS, carrying the human NOS2 gene, or with a control vector, BaglacZ, in which NOS2 is replaced with the β-galactosidase gene (Tzeng, et al., Proc. Natl. Acad. Sci. USA 92:11771-11775 (1995)). Cell clones that constitutively produced nitric oxide were isolated after 14 days of G418 selection (250-350 μg G418/ml). NOS2 and β-galactosidase expressing HCT-116, HT-29, LoVo, RK0 colon carcinoma cells, and Calu-6 lung carcinoma cells (all ATCC, Rockville, Md.), were cultured in A50 medium (Biofluids, Rockville, Md.) supplemented in 10% FBS, 1 mM NG-monomethyl-L-arginine, 5 mM glutamine and 200 μg G418/ml.

Growth rates were determined by plating cells in triplicate dishes at 10³ cells/60 mm dish and staining three dishes per day. Cells were rinsed in phosphate-buffered saline, fixed in 2% formaldehyde and stained with 0.25% crystal violet. The number of cells per colony was determined by counting the stained cells under the microscope. The number of cells was determined in 10 colonies/dish, and population doublings are expressed at log2 (cells/colony).

Determination of nitrite plus nitrate: 3×10⁶ cells were plated in 9 mm² culture wells (Costar, Cambridge, Mass.) and cultured in 4 ml of medium for 48 hr. To determine nitrite plus nitrate concentrations in culture medium, nitrate was converted to nitrite, and nitrite was determined with the Griess reagent (Forrester et al., Proc. Nat'l Acad. Sci USA 93:2442-2447 (1996)).

B. Results

High concentrations of NO induce p53 accumulation and p53-mediated growth arrest and apoptosis (Messmer & Brune, Biochem. J. 319:299-305 (1996); Forrester et al., Proc. Natl. Acad. Sci. USA 93:2442-2447 (1996)). To investigate the functional interaction of p53 and NO in tumor growth, human carcinoma cells, which had a wild-type, missense mutant or p53 null status, were infected with a retroviral construct, DFG-iNOS (Tzeng, et al., Proc. Natl. Acad. Sci USA 92:11771-11775 (1995)). The amounts of NO produced by 10⁶ of these cells ranged from 2 to 15 nmole of nitrite plus nitrate per day (Table 1), which is significantly lower than NO production in cytokine-stimulated macrophages (Lewis et al., J. Biol. Chem. 270:29350-29355 (1995)). Isogenic vector-control carcinoma cell lines, that expressed P-galactosidase (β-gal) instead of human NOS2, did not produce detectable amounts of NO.

TABLE 1
Nitric oxide production* in human carcinoma
cell lines that constitutively express NOS2

| | | | Nitrite plus nitrate |
|---|---|---|---|
| Cell line | | | nmole/day/1 × 10⁶ cells |
| Calu-6 | BaglacZ | | ND |
| | NOS2 | Clone 5 | 8 |
| | NOS2 | Clone 7 | 11 |
| LoVo | BaglacZ | | ND |
| | NOS2 | Clone 9 | 6 |
| RKO | BaglacZ | | ND |
| | NOS2 | Clone 5 | 6 |
| HCT-116 | BaglacZ | | ND |
| | NOS2 | Clone 1 | 2 |
| | NOS2 | Clone 2 | 3 |
| | NOS2 | Clone 3 | 4 |
| HT-29 | BaglacZ | | ND |
| | NOS2 | Clone 1 | 3 |
| | | Clone 2 | 8 |
| | | Clone 3 | 15 |

*Determined as nitrite plus nitrate accumulation in the cell culture medium
ND—not detectable

### Example II

### NOS2 Expression and Tumor Growth

A. Methods Tumor xenoplantation: Suspensions of 3×10⁵ to 5×10⁶ cells in a volume of 0.2 ml were injected at a single subcutaneous site into athymic nude mice previously irradiated with 350 rads. Either 10 or 20 animals were injected per experiment. A nodule was scored as a tumor when it measured 125 mm³ or more by its largest two dimensions.

Statistical Analysis: The Kaplan-Meier survival analysis was used to calculate the statistical significance of tumor probabilities in different treatment groups. Other comparisons were carried out by the two-tailed Student's t-test. Relationships are considered statistically significant when p<0.05.

B. Results

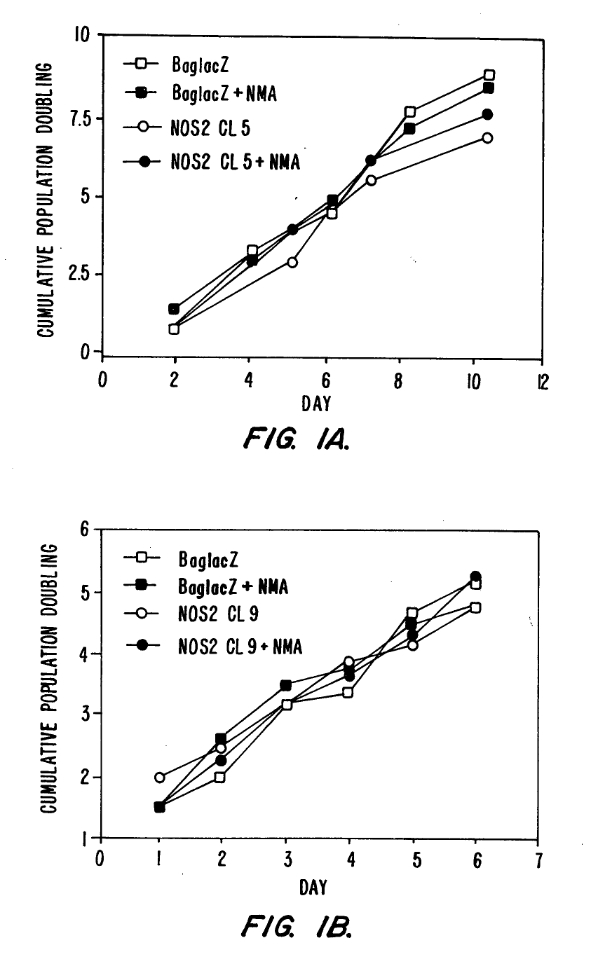

The effects of NOS2 expression on the growth rates of human carcinoma cells were investigated in both in cell culture and in subcutaneous tumors in athymic nude mice. In cell culture, NOS2-expressing carcinoma cell clones grew at the same rate as the isogenic vector controls (FIG. 1). Though NO cytotoxicity has been described in tumor cells after transfection with murine NOS2, it was not observed in the DFG-iNOS infected cell clones, which is consistent with the moderate NO production in these cell lines (Jenkins et al., Proc. Natl. Acad. Sci. U.S.A. 92:4392-4396 (1995); Xie et al., J. Exp. Med. 181:1333-1343 (1995)).

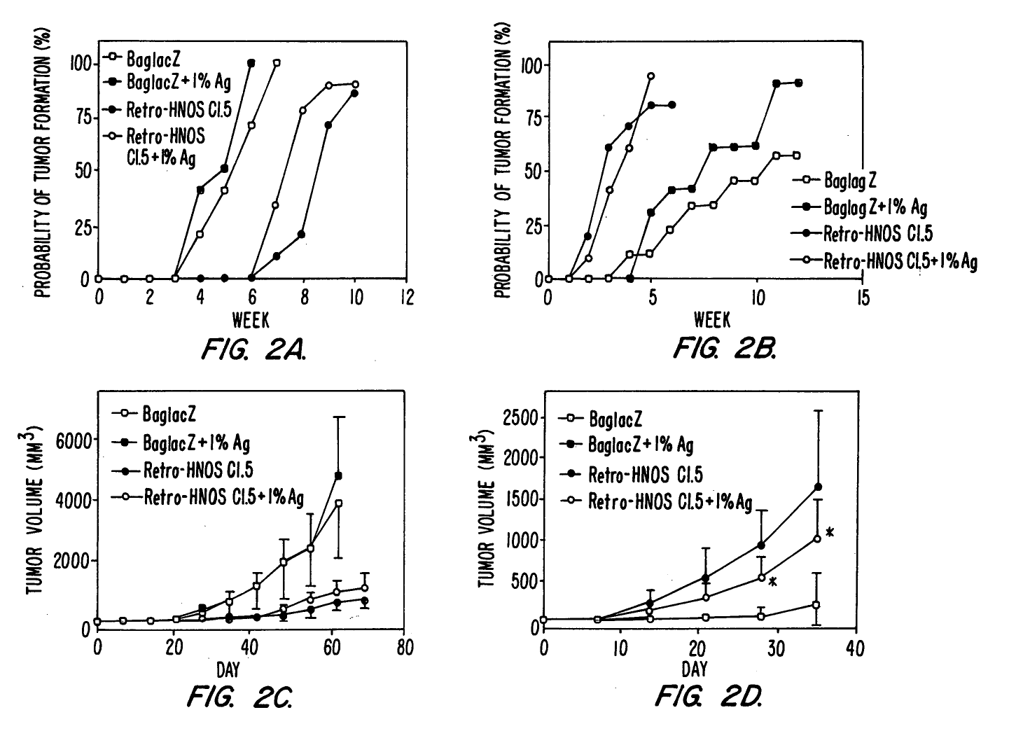

To further evaluate whether NO alters tumor growth, NOS2-or β-gal-expressing carcinoma cells were subcutaneously inoculated into athymic nude mice and tumor growth was monitored. NO-producing LoVo cells that expressed wild-type p53 grew slower and produced smaller tumors than the isogenic vector controls (FIG. 2). In contrast, NO-producing Calu-6 cells that are p53 null grew faster and produced larger tumors than the isogenic vector control cells (FIG. 2).

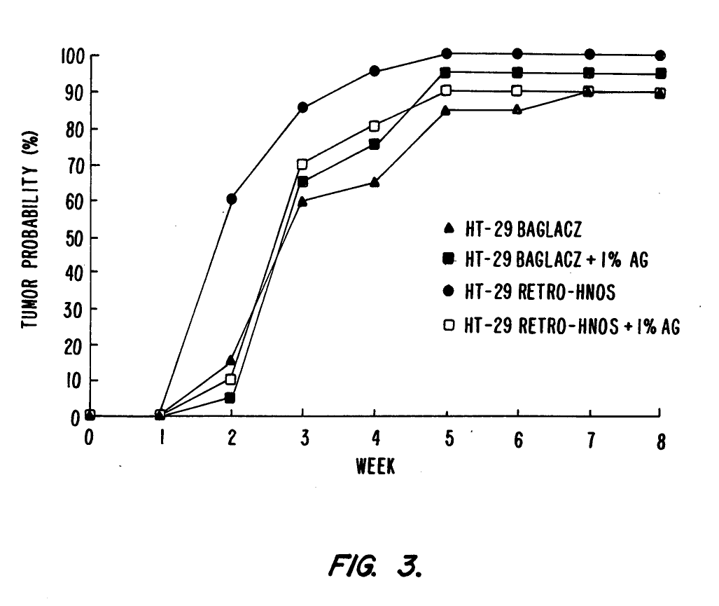

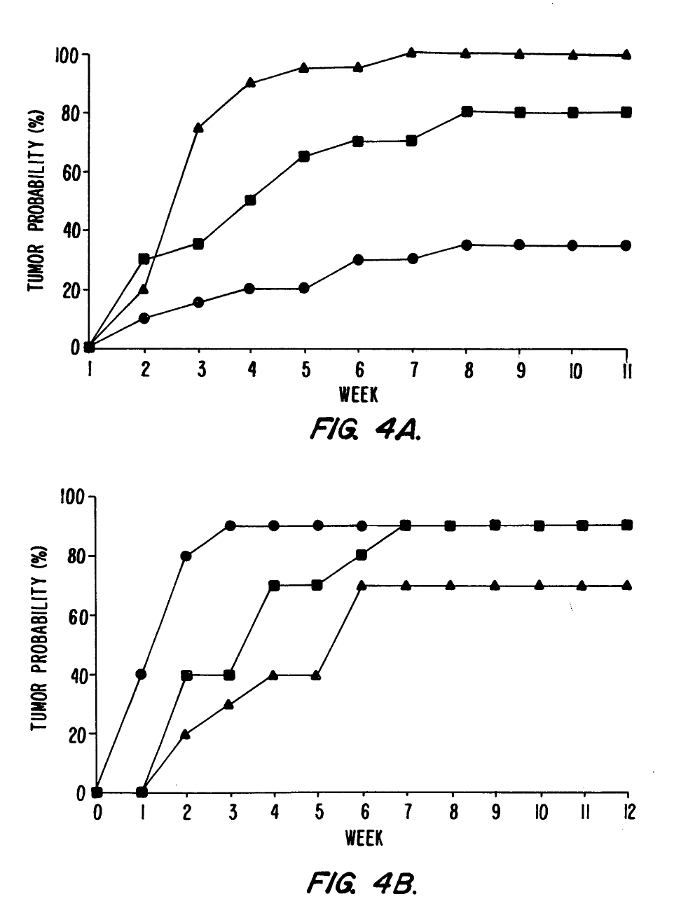

The observations that NO effects tumor growth depending on the p53 status was extended by additional studies. NO affected tumor growth in a dose-dependent manner (FIG. 4), and also reduced the tumor growth of both colon carcinoma cell lines with wild-type p53, RKO and HCT-116 cells, while it accelerated the growth of a colon carcinoma cell line homozygous for mutant p53 (codon 273HIS), HT-29 cells (FIG. 3). The tumors derived from NOS2-expressing LoVo, Calu-6 and RKO cells contained NOS2 activities comparable to those frequently found in a cohort of colorectal tumors and ranged from 3 to 25 pmole/min/mg (Ambs et al., Cancer Res. 58:334-341 (1998)). Furthermore, aminoguanidine, a specific inhibitor of NOS2 (Griffiths et al., Br. J. Pharmacol. 110:963-968 (1993)), significantly reduced the tumor growth of NOS2-expressing Calu-6 (p<0.05, two-tailed Student's t-test, FIG. 2) and HT-29 cells (p=0.002, Kaplan-Meier analysis, FIG. 3)).

### Example III

### NO-Induced Neovascularization

A. Methods

CD31 immunohistochemistry: Five micron sections of ethanol-fixed tumors were deparaffinized and rehydrated, and endogenous peroxidase activity was blocked by treatment of H₂O₂. Sections were incubated with a 1:50 dilution of normal goat serum in PBS/2% BSA and then with the MEC13.3 rat monoclonal anti-mouse CD31 antibody (PharMinger), 1:200 diluted, in PBS/2% BSA for 45 min. Slides were rinsed with PBS and incubated with a secondary, biotin-labeled anti-rat Ig antibody (Vectastain). After incubation with an avidin-biotin-peroxidase complex, slides were stained with 3,3-diaminobenzidine for 10-20 min. The counting of microvessels was performed at ×250 magnification (×25 objective, were scanned and all CD31-positive vessels were counted.

B. Results

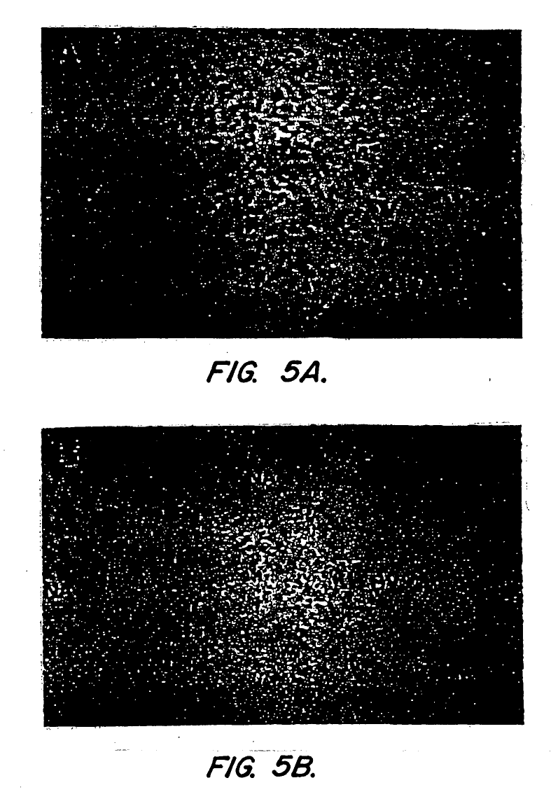

The mechanisms whereby endogenous NO production could accelerate the tumor growth of carcinoma cells which are either null or mutant for p53 were next investigated. NO has angiogenic properties and has been shown to increase the number of blood vessels in tumors grown by DLD-1 human colon carcinoma cells transfected with murine NOS2 (Jenkins et al., Proc. Natl. Acad. Sci. U.S.A. 92:4392-4396 (1995)). Therefore, subcutaneous tumors produced by Calu-6 cells were analyzed in nude mice for angiogenesis by performing immunohistochemistry for CD31, which is a specific marker of endothelial cells and vascularization (Vermeulen et al., Microvasc. Res. 51:164-174 (1996)). Tumors expressing NOS2 contained significantly (p<0.01, two-tailed Student's t-test) more small blood vessels than tumors lacking NOS2 (FIG. 5). Vector control tumors contained large necrotic areas not found in tumors expressing NOS2, and it is likely that deficient angiogenesis limited the growth of these controls. These observations are consistent with reports linking endogenous NO production to an increased tumor growth rate, presumably by enhancing angiogenesis (Jenkins et al., Proc. Natl. Acad. Sci. U.S.A. 92:4392-4396 (1995)). Based on these observations, the lack of an aminoguanidine effect in slow-growing tumors of NOS2-expressing LoVo cells might be explained by insufficient microvascularization, i.e., not allowing an effective inhibitor concentration, while the more vascular tumors of NOS2 expressing Calu-6 cells were inhibited by higher concentrations of aminoguanidine (FIG. 2).

### Example IV

### Increased Vascular Endothelial Growth Factor Expression in NOS2 Expressing Cells

A. Methods

NOS2 and VEGF western blot analysis: Cell lysates for western blotting were prepared by solubilization of cell pellets in RIPA butter. VEGF protein concentrations were determined as follows. Five μg of rabbit polyclonal anti-VEGF antibody (Santa Cruz Biotechnology) were added to either 1 mg of cellular protein extract or 1 ml of cell culture medium, incubated for 1 hr at 8-10° C., and then mixed with protein A-sepharose (10 mg) for 1 hr. Samples were spun at 10,000 g. and pellets were washed with RIPA buffer, boiled in SDS/DTT buffer (5,3-Prime) and loaded on a SDS/13% polyacrylamide gel. For NOS2, 100 μg of soluble protein extract were loaded on a SDS/7% polyacrylamide gel. After transfer to an Immunobilon-P membrane (Millipore), NOS2 and VEGF protein were detected with either a polyclonal anti-NOS2 antibody (Merck), 1:40,000 diluted, or a polyclonal anti-VEGF, 1:1000 diluted, as described (Ambs et al., Cancer Res. 58:334-341 (1998)).

Northern blotting: Total cellular RNA was prepared with the RNeasy™ kit (QIAGEN). 30-50 μg of RNA wee resolved on a 1.2% agarose gel containing 6.3% formaldehyde, transferred to a Hybond™-N nylon membrane (Amersham) and hybridized with a ³²P-labeled cDNA probe containing either the full-length human NOS2 sequence (Geller et al., Proc. Natl. Acad. Sci U.S.A. 90:3491-3495 (1993)) or 522 bp of the human VEGF sequence common for all known VEGF isoforms. The VEGF cDNA was generated by RT-PCR (AdvantageτRT-for-PCR kit, Clontech) using RNA from HCT-116 human colon carcinoma cells. PCR: 32 cycles, 1 min at 58° C., at 72° C. and at 94° C. using Taq polymerase (Perkin Elmer); cDNA primers: 5′-GCCTCCGAAACCATGAACTTTC-3′, 5′-CGAGTCTGTGTTTTTGCAGGAAC-3′.

B. Results

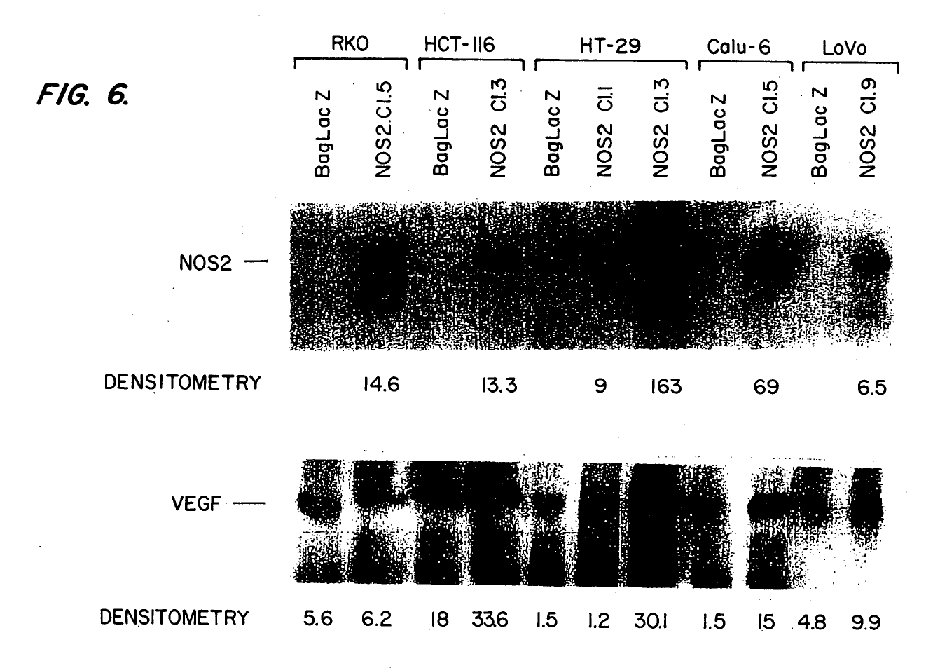

To explore the angiogenic activity of NO, VEGF was investigated as a downstream effector. NO is capable of depleting the intracellular iron storage by which it activates the IRE binding protein (Hentze et al., Proc. Natl. Acad. Sci. U.S.A. 93:8175-8182 (1996)). Iron depletion also activates VEGF expression (Gleadle et al., Am. J. Physiol. 268:C1362-8 (1995)). Therefore, VEGF mRNA and protein expression was investigated in carcinoma cells expressing NOS2. VEGF protein concentrations were higher in cellular extracts of NO expressing clones than in extracts of the vector control cell lines (FIG. 6).

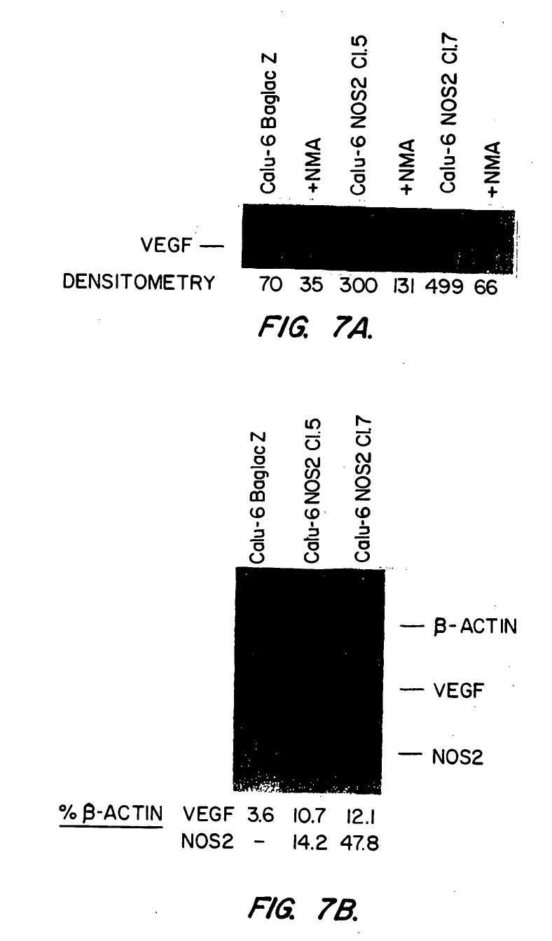

To further confirm this finding, VEGF mRNA levels were determined in Calu-6 cells. VEGF mRNA steady state concentrations were increased in two NOS2-expressing cell clones when compared to the β-gal-expressing vector control (FIG. 7). The VEGF mRNA expression levels also correlated with an increased secretion of VEGF protein into the culture medium (FIG. 7). The addition of a NOS inhibitor, N^{G}-monomethyl-L-arginine (L-NMA), to the cell culture medium reduced the VEGF secretion. These results demonstrate that endogenously produced NO increases VEGF secretion in human carcinoma cells, which is consistent with a recent report showing that NO-donors induce guanylate cyclase-dependent upregulation of VEGF mRNA (Chin et al., Oncogene 15:37-442 (1997)). Additionally, an increased VEGF mRNA level was found in tumors of NOS2-expressing Calu-6 cells.

1-20. (canceled)

21. A method of treating cancer by administering an NOS2 (nitric oxide synthase 2) inhibitor to a patient, the method comprising the steps of:
(i) determining the p53 status of the pateint's cancer or tumor cells; and
(ii) administering an NOS2 inhibitor to the patient when the cancer or tumor cells are p53 mutant cancer or tumor cells.

22. The method of claim 21, wherein the cancer is selected from the group consisting of breast, brain, head, neck, and colon cancer.

23. The method of claim 21, wherein the NOS2 inhibitor is aminoguanidine (AG) or N^{G}-monomethyl-L-arginine (L-NMA).
